SEQUENCE LISTING (13 sequences)

SEQ ID NO: 1 - protein, 576 aa, synthetic construct
MLFKLLQRQTYTCLSHRYGLYVCFLGVVVTIVSAFQFGEVVLEWSRDQYHVLFDSYRDNIAGKSFQNRLCLPMPIDVVYTWVNGTDLELLKELTELKRSKRDPLIPECQGKQTPEKDKCYRDDISASRFEDNEELRYSLRSIERHAPWVRNIFIVTNGQIPSWLNLDNPRVTIVTHQDVFRNLSHLPTFSSPAIESHIHRIEGLSQKFIYLNDDVMFGKDVWPDDFYSHSKGQKVYLTWPVPNGGSGGDTFADSLRYVNKILNSKFGFTSRKVPAHMPHMIDRIVMQELQDMFPEEFDKTSFHKVRHSEDMQFAFSYFYYLMSAVQPLNISQVFDEVDTDQSGVLSDREIRTLATRIHELPLSLQDLTGLEHMLINCSKMLPADITQLNNIPPTQESYYDPNLPPVTKSLVTNCKPVTDKIHKAYKDKNKYRFEIMGEEEIAFKMIRTNVSHVVGQLDDIRKNPRKFVCLNDNIDHNHKDAQTVKAVLRDFYESMFPIPSQFELPREYRNRFLHMHELQEWRAYRDKLKFWTHCVLATLIMFTIFSFFAEQLIALKRKIFPRRRIHKEASPNRIRV

SEQ ID NO: 2 - protein, 547 aa, synthetic construct
MLFKLLQRQTYTCLSHRYGLYVCFLGVVVTIVSAFQFGEVVLEWSRDQYHVLFDSYRDNIAGKSFQNRLCLPMPIDVVYTWVNGTDLELLKELDISASRFEDNEELRYSLRSIERHAPWVRNIFIVTNGQIPSWLNLDNPRVTIVTHQDVFRNLSHLPTFSSPAIESHIHRIEGLSQKFIYLNDDVMFGKDVWPDDFYSHSKGQKVYLTWPVPNGGSGGDTFADSLRYVNKILNSKFGFTSRKVPAHMPHMIDRIVMQELQDMFPEEFDKTSFHKVRHSEDMQFAFSYFYYLMSAVQPLNISQVFDEVDTDQSGVLSDREIRTLATRIHELPLSLQDLTGLEHMLINCSKMLPADITQLNNIPPTQESYYDPNLPPVTKSLVTNCKPVTDKIHKAYKDKNKYRFEIMGEEEIAFKMIRTNVSHVVGQLDDIRKNPRKFVCLNDNIDHNHKDAQTVKAVLRDFYESMFPIPSQFELPREYRNRFLHMHELQEWRAYRDKLKFWTHCVLATLIMFTIFSFFAEQLIALKRKIFPRRRIHKEASPNRIRV

SEQ ID NO: 3 - protein, 552 aa, synthetic construct
MLFKLLQRQTYTCLSHRYGLYVCFLGVVVTIVSAFQFGEVVLEWSRDQYHVLFDSYRDNIAGKSFQNRLCLPMPIDVVYTWVNGTDLELLKELGGGGSDISASRFEDNEELRYSLRSIERHAPWVRNIFIVTNGQIPSWLNLDNPRVTIVTHQDVFRNLSHLPTFSSPAIESHIHRIEGLSQKFIYLNDDVMFGKDVWPDDFYSHSKGQKVYLTWPVPNGGSGGDTFADSLRYVNKILNSKFGFTSRKVPAHMPHMIDRIVMQELQDMFPEEFDKTSFHKVRHSEDMQFAFSYFYYLMSAVQPLNISQVFDEVDTDQSGVLSDREIRTLATRIHELPLSLQDLTGLEHMLINCSKMLPADITQLNNIPPTQESYYDPNLPPVTKSLVTNCKPVTDKIHKAYKDKNKYRFEIMGEEEIAFKMIRTNVSHVVGQLDDIRKNPRKFVCLNDNIDHNHKDAQTVKAVLRDFYESMFPIPSQFELPREYRNRFLHMHELQEWRAYRDKLKFWTHCVLATLIMFTIFSFFAEQLIALKRKIFPRRRIHKEASPNRIRV

SEQ ID NO: 4 - protein, 553 aa, synthetic construct
MLFKLLQRQTYTCLSHRYGLYVCFLGVVVTIVSAFQFGEVVLEWSRDQYHVLFDSYRDNIAGKSFQNRLCLPMPIDVVYTWVNGTDLELLKELGSGSGSDISASRFEDNEELRYSLRSIERHAPWVRNIFIVTNGQIPSWLNLDNPRVTIVTHQDVFRNLSHLPTFSSPAIESHIHRIEGLSQKFIYLNDDVMFGKDVWPDDFYSHSKGQKVYLTWPVPNGGSGGDTFADSLRYVNKILNSKFGFTSRKVPAHMPHMIDRIVMQELQDMFPEEFDKTSFHKVRHSEDMQFAFSYFYYLMSAVQPLNISQVFDEVDTDQSGVLSDREIRTLATRIHELPLSLQDLTGLEHMLINCSKMLPADITQLNNIPPTQESYYDPNLPPVTKSLVTNCKPVTDKIHKAYKDKNKYRFEIMGEEEIAFKMIRTNVSHVVGQLDDIRKNPRKFVCLNDNIDHNHKDAQTVKAVLRDFYESMFPIPSQFELPREYRNRFLHMHELQEWRAYRDKLKFWTHCVLATLIMFTIFSFFAEQLIALKRKIFPRRRIHKEASPNRIRV

SEQ ID NO: 5 - protein, 573 aa, synthetic construct
MLFKLLQRQTYTCLSHRYGLYVCFLGVVVTIVSAFQFGEVVLEWSRDQYHVLFDSYRDNIAGKSFQNRLCLPMPIDVVYTWVNGTDLELLKELGGSGGSPGGSGGSPGGSGGSPGGSGGDISASRFEDNEELRYSLRSIERHAPWVRNIFIVTNGQIPSWLNLDNPRVTIVTHQDVFRNLSHLPTFSSPAIESHIHRIEGLSQKFIYLNDDVMFGKDVWPDDFYSHSKGQKVYLTWPVPNGGSGGDTFADSLRYVNKILNSKFGFTSRKVPAHMPHMIDRIVMQELQDMFPEEFDKTSFHKVRHSEDMQFAFSYFYYLMSAVQPLNISQVFDEVDTDQSGVLSDREIRTLATRIHELPLSLQDLTGLEHMLINCSKMLPADITQLNNIPPTQESYYDPNLPPVTKSLVTNCKPVTDKIHKAYKDKNKYRFEIMGEEEIAFKMIRTNVSHVVGQLDDIRKNPRKFVCLNDNIDHNHKDAQTVKAVLRDFYESMFPIPSQFELPREYRNRFLHMHELQEWRAYRDKLKFWTHCVLATLIMFTIFSFFAEQLIALKRKIFPRRRIHKEASPNRIRV

SEQ ID NO: 6 - protein, 529 aa, synthetic construct
MLFKLLQRQTYTCLSHRYGLYVCFLGVVVTIVSAFQFGEVVLEWSRDQYHVLFDSYRDNIAGKSFQNRLCLPMPIDVVYTWVNGTDLELLKELTELKRSKRDPLIPECQGKQTPEKDKCYRDDISASRFEDNEELRYSLRSIERHAPWVRNIFIVTNGQIPSWLNLDNPRVTIVTHQDVFRNLSHLPTFSSPAIESHIHRIEGLSQKFIYLNDDVMFGKDVWPDDFYSHSKGQKVYLTWPVPNGGSGGDTFADSLRYVNKILNSKFGFTSRKVPAHMPHMIDRIVMQELQDMFPEEFDKTSFHKVRHSEDMQFAFSYFYYLMSAVQPLNISQVFDEVDTDQSGVLSDREIRTLATRIHELPLSLQDLTGLEHMLINCSKMLPADITQLNNIPPTQESYYDPNLPPVTKSLVTNCKPVTDKIHKAYKDKNKYRFEIMGEEEIAFKMIRTNVSHVVGQLDDIRKNPRKFVCLNDNIDHNHKDAQTVKAVLRDFYESMFPIPSQFELPREYRNRFLHMHELQEWRAYRDKLK

SEQ ID NO: 7 - protein, 553 aa, synthetic construct
METDTLLLWVLLLWVPGSTGDSRDQYHVLFDSYRDNIAGKSFQNRLCLPMPIDVVYTWVNGTDLELLKELTELKRSKRDPLIPECQGKQTPEKDKCYRDDISASRFEDNEELRYSLRSIERHAPWVRNIFIVTNGQIPSWLNLDNPRVTIVTHQDVFRNLSHLPTFSSPAIESHIHRIEGLSQKFIYLNDDVMFGKDVWPDDFYSHSKGQKVYLTWPVPNGGSGGDTFADSLRYVNKILNSKFGFTSRKVPAHMPHMIDRIVMQELQDMFPEEFDKTSFHKVRHSEDMQFAFSYFYYLMSAVQPLNISQVFDEVDTDQSGVLSDREIRTLATRIHELPLSLQDLTGLEHMLINCSKMLPADITQLNNIPPTQESYYDPNLPPVTKSLVTNCKPVTDKIHKAYKDKNKYRFEIMGEEEIAFKMIRTNVSHVVGQLDDIRKNPRKFVCLNDNIDHNHKDAQTVKAVLRDFYESMFPIPSQFELPREYRNRFLHMHELQEWRAYRDKLKFWTHCVLATLIMFTIFSFFAEQLIALKRKIFPRRRIHKEASPNRIRV

SEQ ID NO: 8 - protein, 500 aa, synthetic construct
MLFKLLQRQTYTCLSHRYGLYVCFLGVVVTIVSAFQFGEVVLEWSRDQYHVLFDSYRDNIAGKSFQNRLCLPMPIDVVYTWVNGTDLELLKELDISASRFEDNEELRYSLRSIERHAPWVRNIFIVTNGQIPSWLNLDNPRVTIVTHQDVFRNLSHLPTFSSPAIESHIHRIEGLSQKFIYLNDDVMFGKDVWPDDFYSHSKGQKVYLTWPVPNGGSGGDTFADSLRYVNKILNSKFGFTSRKVPAHMPHMIDRIVMQELQDMFPEEFDKTSFHKVRHSEDMQFAFSYFYYLMSAVQPLNISQVFDEVDTDQSGVLSDREIRTLATRIHELPLSLQDLTGLEHMLINCSKMLPADITQLNNIPPTQESYYDPNLPPVTKSLVTNCKPVTDKIHKAYKDKNKYRFEIMGEEEIAFKMIRTNVSHVVGQLDDIRKNPRKFVCLNDNIDHNHKDAQTVKAVLRDFYESMFPIPSQFELPREYRNRFLHMHELQEWRAYRDKLK

SEQ ID NO: 9 - protein, 531 aa, synthetic construct
METDTLLLWVLLLWVPGSTGDEDQVDPRLIDGKGSRPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPANATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 10 - protein, 5 aa, synthetic construct
GGGGS

SEQ ID NO: 11 - protein, 6 aa, synthetic construct
GSGSGS

SEQ ID NO: 12 - protein, 26 aa, synthetic construct
GGSGGSPGGSGGSPGGSGGSPGGSGG

SEQ ID NO: 13 - protein, 29 aa, synthetic construct
TELKRSKRDPLIPECQGKQTPEKDKCYRD

(19) United States
(12) Patent Application Publication
(10) Pub. No.: US 2026/0092263 A1
(43) Pub. Date: Apr. 2, 2026

(54) COMPOSITIONS COMPRISING MODIFIED, TRUNCATED GLCNAC-1-PHOSPHOTRANSFERASE

(71) Applicant: M6P THERAPEUTICS, INC., St. Louis, MO (US)

(72) Inventors: Lin LIU, Ballwin, MO (US); Andrew Charles HEDMAN, St. Louis, MO (US)

(21) Appl. No.: 19/289,364

(22) Filed: Aug. 4, 2025

Related U.S. Application Data
(60) Provisional application No. 63/678,729, filed on Aug. 2, 2024.

(51) Int. Cl. C12N 9/12; C12N 15/864

(52) U.S. Cl. CPC C12N 9/1288; C12N 15/8645; C12Y 207/07049; C12N 2750/14143

(57) ABSTRACT

Provided are amino acid sequences for modified, truncated forms of human GlcNA-1-Phosphotransferase (PTase) that retain phosphotransferase activity and the ability to phosphorylate proteins, lysosomal or non-lysosomal. Truncated forms of PTase lacking or with modified linkers and/or lacking the C-terminal transmembrane and cytosolic domain are demonstrated to retain phosphotransferase activity and the ability to phosphorylate target proteins.

### CROSS REFERENCE TO RELATED APPLICATIONS

This application claims priority to U.S. Patent Application Ser. No. 63/678,729, filed Aug. 2, 2024, the entire contents of which are incorporated herein by reference.

### SEQUENCE LISTING

The instant application contains a Sequence Listing which has been submitted electronically in XML format and is hereby incorporated by reference in its entirety. Said XML copy, created on Dec. 11, 2025, is named P25323US01_SL.xml and is 17,168 bytes in size.

### BACKGROUND

Lysosomal storage disorders (LSDs) relate to inherited metabolic disorders that result from defects in lysosomal function. Currently, about 50 distinct LSDs have been identified but a small number of these (fewer than 10) are reported to have treatments. Many LSDs arise from the lack of activity of a single lysosomal enzyme, which leads to the accumulation of the material normally degraded by the enzyme. Enzyme replacement therapy (ERT) is one promising treatment for LSDs. In ERT, normal lysosomal enzyme is infused intravenously in a LSD patient, taken up via surface mannose 6-phosphate receptors (except β-Glucocerebrosidase for Gaucher disease), and transported to the lysosomes. The feasibility of this approach is dependent upon the ability of the endogenous N-acetylglucosamine-1-phosphotransferase (GlcNAc-1-phosphotransferase) to phosphorylate mannose residues of the N-glycans of the lysosomal enzyme. A few of the replacement enzymes produced by this technique are highly phosphorylated; others, however, are poorly phosphorylated, limiting their effectiveness in ERT. To overcome this limitation, the lysosomal enzyme has been introduced to cells along with wild-type GlcNAc-1-phosphotransferase or a modified GlcNAc-1-phosphotransferase called S1S3 PTase. See Liu et al. 2017, https://doi.org/10.1016/j.omtm.2017.03.006. The disclosure herein provides alternative novel modified and/or truncated forms of GlcNAc-1-phosphotransferase for the safe and effective treatment of LSDs via increased phosphorylation of lysosomal enzymes. The disclosed novel modified and/or truncated forms of GlcNAc-1 phosphotransferase may also be of use for increasing phosphorylation of proteins in general, both lysosomal and non-lysosomal.

### SUMMARY

The present disclosure is directed to a composition comprising a modified truncated form of GlcNAc-1-phosphotransferase that retains the ability to effectively phosphorylate proteins, both lysosomal and non-lysosomal.

In an embodiment, the modified GlcNAc-1-phosphotransferase comprises the amino acid sequence of SEQ ID NO: 1 with a deletion of amino acids 109-137. In some embodiments, the sequence of the modified GlcNAc-1-phosphotransferase comprises the amino acid sequence of SEQ ID NO: 2. In another embodiment, the deletion of amino acids 109-137 is replaced with an amino acid sequence of 5-6 amino acids. In some embodiments, 5-6 amino acids comprises at least three glycines. In some embodiments, the amino acid sequence of 5-6 amino acids comprises glycine-glycine-glycine-glycine-serine or glycine-serine-glycine-serine-glycine-serine. In some embodiments, the amino acid sequence of the modified GlcNAc-1-phosphotransferase comprises the amino acid sequence of SEQ ID NO: 3 or SEQ ID NO: 4. In another embodiment, the deletion of amino acids 109-137 is replaced with an amino acid sequence of 26 amino acids. In some embodiments, the amino acid sequence comprises at least ten glycines. In some embodiments, the amino acid sequence of the modified GlcNAc-1-phosphotransferase comprises the amino acid sequence of SEQ ID NO: 5. In any of the foregoing embodiments, the modified GlcNAc-1-phosphotransferase further comprises a deletion of the C-terminal transmembrane and cytosolic domain. In some embodiments, the deletion of the C-terminal transmembrane and cytosolic domain comprises a deletion of amino acids 530-576 of SEQ ID NO: 1. In some embodiments, the amino acid sequence of the modified GlcNAc-1-phosphotransferase comprises the amino acid sequence of SEQ ID NO: 8.

In an embodiment, the modified GlcNAc-1-phosphotransferase comprises the amino acid sequence of SEQ ID NO: 1 with a deletion of the C-terminal transmembrane and cytosolic domain. In some embodiments, the deletion of the C-terminal transmembrane and cytosolic domain comprises a deletion of amino acids 530-576 S1S3 PTase. In some embodiments, the amino acid sequence of the modified GlcNAc-1-phosphotransferase comprises the amino acid sequence of SEQ ID NO: 6 or SEQ ID NO: 8. In some embodiments, the modified GlcNAc-1-phosphotransferase further comprises a deletion of amino acids 109-137.

The present disclosure is also directed to compositions comprising a vector comprising the modified GlcNA-c-1 phosphotransferases described herein and a protein (lysosomal or non-lysosomal) in which additional phosphorylation is desired.

The present disclosure is also directed to a method of increasing phosphorylation of a protein (lysosomal or non-lysosomal) comprising contacting a cell with the vectors comprising a lysosomal enzyme and the modified GlcNA-c-1 phosphotransferases described herein.

The present disclosure is also directed to a method of treating a lysosomal storage disorder comprising administering to a subject the vectors comprising a lysosomal enzyme and the modified GlcNA-c-1 phosphotransferases described herein.

### BRIEF DESCRIPTION OF THE DRAWINGS

For the purpose of illustrating the disclosure, there are depicted in the drawings certain embodiments of the disclosure. However, the disclosure is not limited to the precise arrangements and instrumentalities of the embodiments depicted in the drawings.

FIG. 1A is a graphical depiction of the S1S3 constructs for investigation of different linker sequences. Figure discloses SEQ ID NOS 13 and 10-12, respectively, in order of appearance.

FIG. 1B is a western blot showing expression of the S1S3 constructs of FIG. 1A in HEK293T cells.

FIG. 1C depicts the PTase activity of the S1S3 constructs of FIG. 1A.

FIG. 2 is a graphical depiction of the S1S3 constructs for investigation of constructs with removal of the linker sequence and/or transmembrane domains.

FIG. 3A depicts PTase activity of the S1S3 constructs of FIG. 2.

FIG. 3B is a western blot showing expression of the S1S3 constructs of FIG. 2 in Expi293 cells.

FIG. 4 shows immunofluorescence images of permeabilized HeLa cells transfected with the S1S3 plasmid with v5 tag (G0047, SEQ ID NO: 1) compared with variants in which the transmembrane region was removed at the C-terminal (G0067, SEQ ID NO: 6) or N-terminal (G0068, SEQ ID NO: 7). S1S3 and the variants were probed with an anti-V5 mouse monoclonal antibody (green) and colocalized with the Golgi marker giantin.

FIG. 5 depicts the binding GCase enzyme to the CI-MPR in conditioned media from Expi293 cells transfected with the S1S3 constructs of FIG. 2.

FIG. 6A depicts a schematic representation of exemplary truncated versions of S1S3 incorporating the GBA1 gene.

FIG. 6B depicts a graph showing GCase activity in conditioned media from transfected cells.

FIG. 6C depicts a Western Blot showing GCase expression from cell lysates of transfected cells.

FIG. 6D depicts a graph showing binding of GCase to CI-MPR in conditioned media from transfected cells.

FIG. 7 shows the experimental protocol for an expression study of AAV constructs containing the GBA1 gene and truncated S1S3.

FIG. 8A depicts GBA1 gene copies in liver tissue from mice transduced with AAV-GBA1-truncated S1S3 constructs.

FIG. 8B depicts GBA1 mRNA levels in liver tissue from mice transduced with AAV-GBA1-truncated S1S3 constructs.

FIG. 8C depicts GBA1 gene copies in heart tissue from mice transduced with AAV-GBA1-truncated S1S3 constructs.

FIG. 8D depicts GBA1 mRNA levels in heart tissue from mice transduced with AAV-GBA1-truncated S1S3 constructs.

FIG. 8E depicts GBA1 gene copies in brain tissue from mice transduced with AAV-GBA1-truncated S1S3 constructs.

FIG. 8F depicts GBA1 mRNA levels in brain tissue from mice transduced with AAV-GBA1-truncated S1S3 constructs.

FIG. 8G depicts GBA1 gene copies in bone marrow tissue from mice transduced with AAV-GBA1-truncated S1S3 constructs.

FIG. 8H depicts GBA1 mRNA levels in bone marrow tissue from mice transduced with AAV-GBA1-truncated S1S3 constructs.

FIG. 8I depicts GBA1 gene copies in spleen tissue from mice transduced with AAV-GBA1-truncated S1S3 constructs.

FIG. 8J depicts GBA1 mRNA levels in spleen tissue from mice transduced with AAV-GBA1-truncated S1S3 constructs.

FIG. 9A depicts total GCase produced in serum for wild type mice transduced with AAV-GBA1-truncated S1S3 constructs.

FIG. 9B depicts GCase activity in liver tissue from the wild type mice transduced with AAV-GBA1-truncated S1S3 constructs.

FIG. 9C depicts GCase activity in heart tissue from the wild type mice transduced with AAV-GBA1-truncated S1S3 constructs.

FIG. 9D depicts GCase activity in brain tissue from the wild type mice transduced with AAV-GBA1-truncated S1S3 constructs.

FIG. 10 shows the experimental protocol for an expression and biodistribution study in the brain tissue of mice transduced with AAV constructs containing the GBA1 gene and truncated S1S3.

FIG. 11 shows the results of the expression and biodistribution study in the brain tissue of mice. The top panels show increased human GCase protein in mouse brain sections detected by anti-human GCase antibody. The bottom panels show staining indicative of the presence of human GBA mRNA.

FIG. 12A depicts experimental results showing GCase protein expression in sections of the brain tissues of mice.

FIG. 12B depicts the pixel intensity for human GCase positive staining that was quantified in hind brain tissue. FIG. 12C depicts the pixel intensity for human GCase positive staining that was quantified in visual cortex tissue.

FIG. 12D depicts the pixel intensity for human GCase positive staining that was quantified in somatosensory cortex tissue.

FIG. 12E depicts the pixel intensity for human GCase positive staining that was quantified in cerebellum tissue.

FIG. 12F depicts the pixel intensity for human GCase positive staining that was quantified in midbrain tissue.

FIG. 12G depicts the pixel intensity for human GCase positive staining that was quantified in olfactory bulb tissue.

FIG. 12H depicts the pixel intensity for human GCase positive staining that was quantified in thalamus tissue.

### DETAILED DESCRIPTION

The present disclosure is directed to novel modified S1S3 variants of GlcNA-1-Phosphotransferase (S1S3 PTase). S1S3 PTase is disclosed in PCT Publication No. WO 2021003442A1, the entirety of which is incorporated herein by reference.

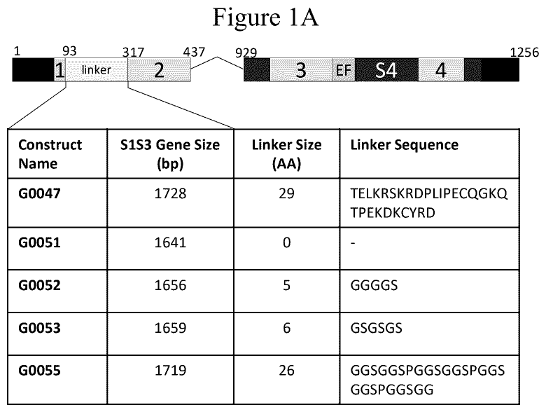

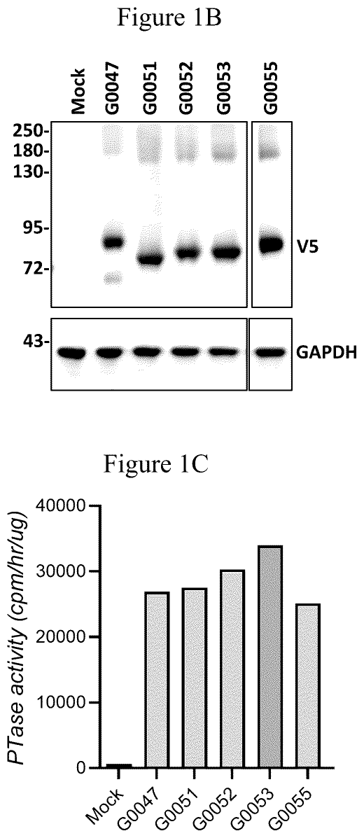

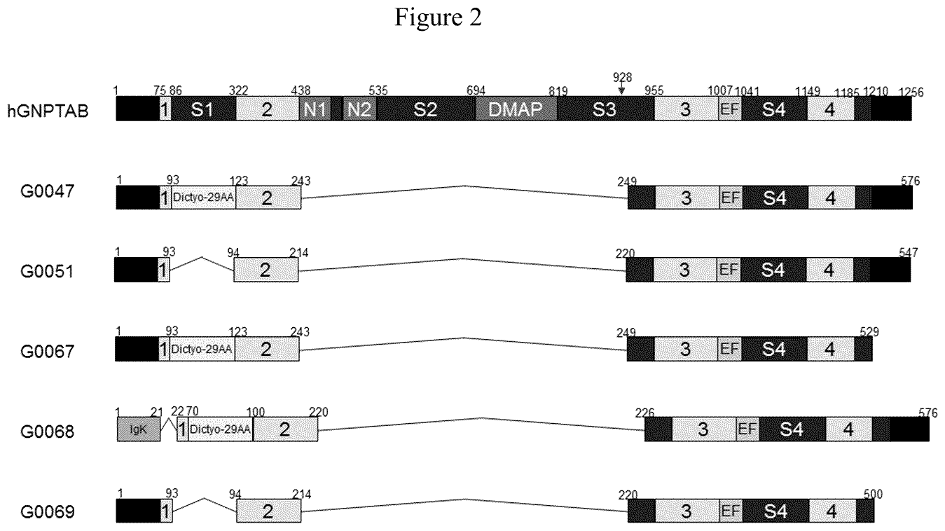

Disclosed herein are modified and truncated versions of S1S3 PTase to create shorter forms of the enzyme that retain phosphotransferase activity and the capability to phosphorylate proteins, both lysosomal and non-lysosomal. Based on the coding sequence for human N-acetylglucosamine-1-phosphate transferase subunits alpha and beta (GNPTAB gene, NM_024312.5) these modifications alter the S1S3 sequence (SEQ ID NO: 4 of WO2021003442) to: 1) remove the dictyostelium linker and replace it with a flexible glycine/serine (Gly/Ser) linker; and 2) remove transmembrane and/or cytosolic domains. FIG. 1A shows representative Gly/Ser linker substitutions having between 0-26 amino acids. FIG. 2 depicts the original S1S3 PTase (G0047) having the dictyostelium linker (shown as Dictyo-29AA), compared with truncated constructs in which the dictyostelium sequence and/or transmembrane or cytosolic domains have been removed.

The modified truncated versions of S1S3 PTase may be used to phosphorylate a lysosomal protein. In some embodiments, the protein is involved in at least one lysosomal storage disorder (LSD) as listed in Table 1A, Table IB or Table 1C of WO20210033442A1. In some embodiments, the lysosomal protein comprises at least one lysosomal enzyme listed in Table 1 A, Table IB or Table 1C of WO20210033442A1, which is incorporated herein by reference.

In embodiments in which the protein is a lysosomal protein, the lysosomal protein is selected from the group consisting of β-glucocerebrosidase (GCase/GBA, encoded by the GBA gene), galactosylceramidase (GALC), α-galactosidase (encoded by the GLA gene), α-N-acetylglucosaminidase (NAGLU), acid α-glucosidase (GAA) and lysosomal acid a-mannosidase (LAMAN).

In some embodiments, the non-lysosomal protein comprises, for example, a non-lysosomal protein or a polypeptide fragment of a non-lysosomal protein, including, without limitation, a cytokine, a membrane receptor, or immune checkpoint molecule. In some embodiments, the non-lysosomal protein is selected from Tumor necrosis factor-α (TNFα), interferons, interleukins, IL-2, IL-12, growth IGF, EGF, EGFR, VEGF, insulin, PD-L1, PD-1, and adrenaline.

The modified truncated versions of S1S3 PTase may be used in a method of treating a lysosomal storage disorder (LSD) as described in WO20210033442A1, which is incorporated herein by reference. The method comprises administering to a subject an effective amount of a composition of the disclosure, wherein the composition increases the phosphorylation of a lysosomal enzyme responsible of the LSD, thereby treating the LSD. The compositions of the present invention increase the N-linked oligosaccharide phosphorylation of a lysosomal enzyme responsible of the LSD, thereby treating the LSD.

In some embodiments, a nucleic acid construct comprising the present modified or truncated versions of S1S3 may further comprise an expression vector, as described in WO20210033442A1. In some embodiments, the expression vector comprises a plasmid.

In some embodiments, the expression vector is a delivery vector, such as a viral vector. In some embodiments, the viral vector comprises an AAV vector or a lentiviral vector. In some embodiments, the AAV vector comprises a sequence isolated or derived from an AAV of serotype AAV1, AAV2, AAV3, AAV4, AAV5, AAV6, AAV7, AAV8 or AAV9.

In some embodiments, the delivery vector comprises a non-viral vector. In some embodiments the non-viral vector comprises a liposome, a lipid nanoparticle, (LNP), a micelle, a polymersome, a nanoparticle, a polymer nanoparticle, or an exosome.

The invention disclosed herein is not limited to the GCase protein; rather, the invention disclosed herein may comprise any other protein, lysosomal or non-lysosomal together with the truncated or modified S1S3 PTase. Use of GCase in the examples below is exemplary and not intended to limit the invention.

As demonstrated in the following Examples, the truncated forms of S1S3 PTase disclosed herein evidence phosphotransferase activity similar to or better than S1S3 PTase.

### EXAMPLES

The disclosure is described with reference to the following Examples. These Examples are provided for the purpose of illustration only and the disclosure should in no way be construed as being limited to these Examples, but rather should be construed to encompass any and all variations which become evident as a result of the disclosure provided herein.

Without further description, it is believed that one of ordinary skill in the art can, using the preceding description and the following illustrative examples, make and utilize the compounds of the present disclosure and practice the claimed methods. The following working examples are not to be construed as limiting in any way the present disclosure.

### Example 1

Construction of Modified GlcNAc-1-phosphotransferase.

S1S3 plasmid with v5 tag (encoding S1S3 PTase amino acid sequence G0047, SEQ ID NO: 1) was modified by PCR and gene fragments using standard molecular biology techniques. To change the linker sequence, gene fragments were ordered from Twist Biosciences (https://www.twistbioscience.com/) that delete the dictyostelium peptide (29 amino acids (“AA”)) sequence (AA109-137 from XP_638036.1) that was placed between the human GNPTAB (AA 94-316). The dictyostelium peptide was deleted or replaced with flexible Gly/Ser linkers (see, e.g., https://doi.org/10.1016/bs.mie.2020.12.001; doi: 10.1021/bi061288t; doi: 10.1073/pnas.95.11.5929) containing: 1) zero amino acids [G0051, SEQ ID NO: 2], 2) 5 amino acids (GGGGS (SEQ ID NO: 10)) [G0052, SEQ ID NO: 3], 3) 6 amino acids (GSGSGS (SEQ ID NO: 11)) [G0053, SEQ ID NO: 4] or 4) 26 amino acids (GGSGGSPGGSGGSPGGSGGSPGGSGG (SEQ ID NO: 12)) [G0055, SEQ ID NO: 5]. Using NheI and XhoI restriction sites and standard ligation techniques. S1S3—with modified linkers were expressed in a pcDNA3.1 vector with a c-terminal v5 tag. Sequences were confirmed with Sanger sequencing. Expression was detected using an anti-v5 tag antibody.

(G0047) (576 AA)
SEQ ID NO: 1
MLFKLLQRQTYTCLSHRYGLYVCFLGVVVTIVSAFQFGEVVLEWS
RDQYHVLFDSYRDNIAGKSFQNRLCLPMPIDVVYTWVNGTDLELL
KELTELKRSKRDPLIPECQGKQTPEKDKCYRDDISASRFEDNEEL
RYSLRSIERHAPWVRNIFIVTNGQIPSWLNLDNPRVTIVTHQDVF
RNLSHLPTFSSPAIESHIHRIEGLSQKFIYLNDDVMFGKDVWPDD
FYSHSKGQKVYLTWPVPNGGSGGDTFADSLRYVNKILNSKFGFTS
RKVPAHMPHMIDRIVMQELQDMFPEEFDKTSFHKVRHSEDMQFAF
SYFYYLMSAVQPLNISQVFDEVDTDQSGVLSDREIRTLATRIHEL
PLSLQDLTGLEHMLINCSKMLPADITQLNNIPPTQESYYDPNLPP
VTKSLVTNCKPVTDKIHKAYKDKNKYRFEIMGEEEIAFKMIRTNV
SHVVGQLDDIRKNPRKFVCLNDNIDHNHKDAQTVKAVLRDFYESM
FPIPSQFELPREYRNRFLHMHELQEWRAYRDKLKFWTHCVLATLI
MFTIFSFFAEQLIALKRKIFPRRRIHKEASPNRIRV
(G0051) (547 AA)
SEQ ID NO: 2
MLFKLLQRQTYTCLSHRYGLYVCFLGVVVTIVSAFQFGEVVLEWS
RDQYHVLFDSYRDNIAGKSFQNRLCLPMPIDVVYTWVNGTDLELL
KELDISASRFEDNEELRYSLRSIERHAPWVRNIFIVTNGQIPSWL
NLDNPRVTIVTHQDVFRNLSHLPTFSSPAIESHIHRIEGLSQKFI
YLNDDVMFGKDVWPDDFYSHSKGQKVYLTWPVPNGGSGGDTFADS
LRYVNKILNSKFGFTSRKVPAHMPHMIDRIVMQELQDMFPEEFDK
TSFHKVRHSEDMQFAFSYFYYLMSAVQPLNISQVFDEVDTDQSGV
LSDREIRTLATRIHELPLSLQDLTGLEHMLINCSKMLPADITQLN
NIPPTQESYYDPNLPPVTKSLVTNCKPVTDKIHKAYKDKNKYRFE
IMGEEEIAFKMIRTNVSHVVGQLDDIRKNPRKFVCLNDNIDHNHK
DAQTVKAVLRDFYESMFPIPSQFELPREYRNRFLHMHELQEWRAY
RDKLKFWTHCVLATLIMFTIFSFFAEQLIALKRKIFPRRRIHKEA
SPNRIRV
(G0052) (552 AA)
SEQ ID NO: 3
MLFKLLQRQTYTCLSHRYGLYVCFLGVVVTIVSAFQFGEVVLEWS
RDQYHVLFDSYRDNIAGKSFQNRLCLPMPIDVVYTWVNGTDLELL
KELGGGGSDISASRFEDNEELRYSLRSIERHAPWVRNIFIVTNGQ
IPSWLNLDNPRVTIVTHQDVFRNLSHLPTFSSPAIESHIHRIEGL
SQKFIYLNDDVMFGKDVWPDDFYSHSKGQKVYLTWPVPNGGSGGD
TFADSLRYVNKILNSKFGFTSRKVPAHMPHMIDRIVMQELQDMFP
EEFDKTSFHKVRHSEDMQFAFSYFYYLMSAVQPLNISQVFDEVDT
DQSGVLSDREIRTLATRIHELPLSLQDLTGLEHMLINCSKMLPAD
ITQLNNIPPTQESYYDPNLPPVTKSLVTNCKPVTDKIHKAYKDKN
KYRFEIMGEEEIAFKMIRTNVSHVVGQLDDIRKNPRKFVCLNDNI
DHNHKDAQTVKAVLRDFYESMFPIPSQFELPREYRNRFLHMHELQ
EWRAYRDKLKFWTHCVLATLIMFTIFSFFAEQLIALKRKIFPRRR
IHKEASPNRIRV
(G0053) (553 AA)
SEQ ID NO: 4
MLFKLLQRQTYTCLSHRYGLYVCFLGVVVTIVSAFQFGEVVLEWS
RDQYHVLFDSYRDNIAGKSFQNRLCLPMPIDVVYTWVNGTDLELL
KELGSGSGSDISASRFEDNEELRYSLRSIERHAPWVRNIFIVING
QIPSWLNLDNPRVTIVTHQDVFRNLSHLPTFSSPAIESHIHRIEG
LSQKFIYLNDDVMFGKDVWPDDFYSHSKGQKVYLTWPVPNGGSGG
DTFADSLRYVNKILNSKFGFTSRKVPAHMPHMIDRIVMQELQDMF
PEEFDKTSFHKVRHSEDMQFAFSYFYYLMSAVQPLNISQVFDEVD
TDQSGVLSDREIRTLATRIHELPLSLQDLTGLEHMLINCSKMLPA
DITQLNNIPPTQESYYDPNLPPVTKSLVTNCKPVTDKIHKAYKDK
NKYRFEIMGEEEIAFKMIRTNVSHVVGQLDDIRKNPRKFVCLNDN
IDHNHKDAQTVKAVLRDFYESMFPIPSQFELPREYRNRFLHMHEL
QEWRAYRDKLKFWTHCVLATLIMFTIFSFFAEQLIALKRKIFPRR
RIHKEASPNRIRV
(G0055) (573 AA)
SEQ ID NO: 5
MLFKLLQRQTYTCLSHRYGLYVCFLGVVVTIVSAFQFGEVVLEWS
RDQYHVLFDSYRDNIAGKSFQNRLCLPMPIDVVYTWVNGTDLELL
KELGGSGGSPGGSGGSPGGSGGSPGGSGGDISASRFEDNEELRYS
LRSIERHAPWVRNIFIVTNGQIPSWLNLDNPRVTIVTHQDVFRNL
SHLPTFSSPAIESHIHRIEGLSQKFIYLNDDVMFGKDVWPDDFYS
HSKGQKVYLTWPVPNGGSGGDTFADSLRYVNKILNSKFGFTSRKV
PAHMPHMIDRIVMQELQDMFPEEFDKTSFHKVRHSEDMQFAFSYF
YYLMSAVQPLNISQVFDEVDTDQSGVLSDREIRTLATRIHELPLS
LQDLTGLEHMLINCSKMLPADITQLNNIPPTQESYYDPNLPPVTK
SLVTNCKPVTDKIHKAYKDKNKYRFEIMGEEEIAFKMIRTNVSHV
VGQLDDIRKNPRKFVCLNDNIDHNHKDAQTVKAVLRDFYESMFPI
PSQFELPREYRNRFLHMHELQEWRAYRDKLKFWTHCVLATLIMFT
IFSFFAEQLIALKRKIFPRRRIHKEASPNRIRV

In addition, primers were used to amplify a region of S1S3 to remove the 3′-transmembrane domain (after human GNPTAB amino acid 1209). Additional constructs were made to remove the transmembrane regions found at the N or C terminus. These constructs that remove the C-terminal transmembrane (G0067, SEQ ID NO: 6), N-terminal transmembrane (G0068, SEQ ID NO: 7) or C-terminal transmembrane and 29 AA dictyostelium sequence (G0069, SEQ ID NO: 8) were also tested.

(G0067) (529 AA)
SEQ ID NO: 6
MLFKLLQRQTYTCLSHRYGLYVCFLGVVVTIVSAFQFGEVVLEWS
RDQYHVLFDSYRDNIAGKSFQNRLCLPMPIDVVYTWVNGTDLELL
KELTELKRSKRDPLIPECQGKQTPEKDKCYRDDISASRFEDNEEL
RYSLRSIERHAPWVRNIFIVINGQIPSWLNLDNPRVTIVTHQDVF
RNLSHLPTFSSPAIESHIHRIEGLSQKFIYLNDDVMFGKDVWPDD
FYSHSKGQKVYLTWPVPNGGSGGDTFADSLRYVNKILNSKFGFTS
RKVPAHMPHMIDRIVMQELQDMFPEEFDKTSFHKVRHSEDMQFAF
SYFYYLMSAVQPLNISQVFDEVDTDQSGVLSDREIRTLATRIHEL
PLSLQDLTGLEHMLINCSKMLPADITQLNNIPPTQESYYDPNLPP
VTKSLVTNCKPVTDKIHKAYKDKNKYRFEIMGEEEIAFKMIRTNV
SHVVGQLDDIRKNPRKFVCLNDNIDHNHKDAQTVKAVLRDFYESM
FPIPSQFELPREYRNRFLHMHELQEWRAYRDKLK
(G0068) (576 AA)
SEQ ID NO: 7
METDTLLLWVLLLWVPGSTGDSRDQYHVLFDSYRDNIAGKSFQNR
LCLPMPIDVVYTWVNGTDLELLKELTELKRSKRDPLIPECQGKQT
PEKDKCYRDDISASRFEDNEELRYSLRSIERHAPWVRNIFIVING
QIPSWLNLDNPRVTIVTHQDVFRNLSHLPTFSSPAIESHIHRIEG
LSQKFIYLNDDVMFGKDVWPDDFYSHSKGQKVYLTWPVPNGGSGG
DTFADSLRYVNKILNSKFGFTSRKVPAHMPHMIDRIVMQELQDMF
PEEFDKTSFHKVRHSEDMQFAFSYFYYLMSAVQPLNISQVFDEVD
TDQSGVLSDREIRTLATRIHELPLSLQDLTGLEHMLINCSKMLPA
DITQLNNIPPTQESYYDPNLPPVTKSLVTNCKPVTDKIHKAYKDK
NKYRFEIMGEEEIAFKMIRTNVSHVVGQLDDIRKNPRKFVCLNDN
IDHNHKDAQTVKAVLRDFYESMFPIPSQFELPREYRNRFLHMHEL
QEWRAYRDKLKFWTHCVLATLIMFTIFSFFAEQLIALKRKIFPRR
RIHKEASPNRIRV
(G0069) (500 AA)
SEQ ID NO: 8
MLFKLLQRQTYTCLSHRYGLYVCFLGVVVTIVSAFQFGEVVLEWS
RDQYHVLFDSYRDNIAGKSFQNRLCLPMPIDVVYTWVNGTDLELL
KELDISASRFEDNEELRYSLRSIERHAPWVRNIFIVTNGQIPSWL
NLDNPRVTIVTHQDVFRNLSHLPTFSSPAIESHIHRIEGLSQKFI
YLNDDVMFGKDVWPDDFYSHSKGQKVYLTWPVPNGGSGGDTFADS
LRYVNKILNSKFGFTSRKVPAHMPHMIDRIVMQELQDMFPEEFDK
TSFHKVRHSEDMQFAFSYFYYLMSAVQPLNISQVFDEVDTDQSGV
LSDREIRTLATRIHELPLSLQDLTGLEHMLINCSKMLPADITQLN
NIPPTQESYYDPNLPPVTKSLVTNCKPVTDKIHKAYKDKNKYRFE
IMGEEEIAFKMIRTNVSHVVGQLDDIRKNPRKFVCLNDNIDHNHK
DAQTVKAVLRDFYESMFPIPSQFELPREYRNRFLHMHELQEWRAY
RDKLK

### Example 2

### Methods.

Cell culture and transfection: HEK293T cells grown in DMEM (Sigma, Cat #D6429) plus 10% FBS were cultured in 12 well dishes and transfected using Lipofectamine 3000 reagent (Invitrogen, Cat #3000015) and (Opti-MEM (Gibco, Cat #31985-070) following manufacturer's protocol. Cell lysate was collected 48 h after transfection. Cells were rinsed in PBS, lysed in M-PER buffer (Thermo 78501) containing 1% protease and phosphatase inhibitor cocktail (MidSci IB01070), sonicated, and centrifuged to remove debris. Cell lysate was then examined by Western blot and phosphotransferase (PTase) activity assay.

PTase assay protocol: See Liu et al 2017, https://doi.org/10.1016/j.omtm.2017.03.006.

SDS-PAGE and Western Blotting: Samples were boiled in protein loading buffer and examined on NuPage 4-12% Gradient Bis-Tris polyacrylamide gels. Gels were then stained using Coomassie Stain or transferred to nitrocellulose membrane. Blots were blocked in 5% Milk in PBST. Blots were probed with anti-v5 tag antibodies (Invitrogen, 46-0705) (updated to R960-25) and HRP-tagged sheep anti-mouse secondary antibodies (ECL, NA931V). Blots were incubated with ECL substrate. Images of Coomassie stained gels or Western blots were captured using an Azure 400 Imaging System.

Cell culture and transfection: Expi293F cells (ThermoFisher, A14527) were cultured under the Expi293 expression medium following the instruction. Cell transfection was performed with ExpiFectamine 293 transfection kit (ThermoFisher, A14524) following the instructions.

Conditioned medium and cell harvesting: Conditioned medium from transfected cells was harvested by two times centrifugation. Generally, 1 mL cells were harvested by spinning down the cells at 500 g for 5 minutes. Supernatant—the conditioned medium was transferred to a new 1.7 mL EP tube and centrifuge again at 16,000 g for 10 min. the supernatant was saved at −80 for future enzyme activity, western blotting and CI-MPR binding analysis. The cell pellet was washed once with PBS buffer by pipetting up and down three times. Then spin down again at 500 g for 5 minutes. Supernatant PBS was removed, and cell pellet was saved at −80 C.

Cell extraction: The cell pellet was pulled out from −80 C and the following was added: 500-1000 uL ice-cold 50 nM tris-Cl buffer, pH7.4, 120 mM NaCl, 1% Triton-100 with 1% Protease inhibitor cocktail. Then the pellet was sonicated at 50% power for 6 sec, put on ice for 10 minutes, and centrifuged at 16,000 g for 10 min. The clear supernatant was transferred to a new EP tube.

### Example 3

Function of S1S3 PTase Variants with Different Linker Sequences.

S1S3 PTase (G0047; previously described in Liu et al 2017, https://doi.org/10.1016/j.omtm.2017.03.006) was modified to replace a 29 AA linker sequence with linkers of 0, 5, 6 or 26 AA (named G0051, G0052, G0053 and G0055 respectively). FIG. 1A shows the different linker constructs 1) G0047—dictyostelium peptide linker, 2) G0051—No linker, 3) G0052-5 amino acid linker (GGGGS (SEQ ID NO: 10)), 4) G0053—6 amino acid linker (GSGSGS (SEQ ID NO: 11)) and 5) G0055—26 amino acid linker (GGSGGSPGGSGGSPGGSGGSPGGSGG (SEQ ID NO: 12)). Each S1S3 PTase with modified linker enzyme was expressed alone with a C-terminal V5 tag by transfection of HEK293T cells. These were compared to the expression of the original S1S3 construct containing 29 AA peptide from dictyostelium. Western blotting of cell lysates with a v5 antibody shows similar expression of the S1S3 with varying linker inserts (FIG. 1B). PTase activity assay shows that the versions of S1S3 with varying linkers all retain PTase activity (FIG. 1C). The data suggests that the linker sequence does not affect the S1S3 enzyme activity, and the removal of the linker sequence provides us an opportunity for smaller size S1S3 gene.

### Example 4

Function of S1S3 PTase Variants Lacking Linker and/or N-Terminal or C-Terminal Domains.

To further investigate the role of transmembrane and cytosolic domains, a serial construct to remove the N-terminal cytosol and transmembrane domain or the C-terminal cytosol and transmembrane domain as described in Example 1 was generated. FIG. 2 shows the different S1S3 PTase variants that remove transmembrane domains or dictyostelium linker region—1) G0047—dictyostelium peptide linker remains with both transmembrane domains intact, 2) G0051—dictyostelium peptide linker removed, 3) G0067-dictyostelium peptide linker remains and C terminal cytosol tail and C terminal transmembrane domain removed, 4) G0068—dictyostelium peptide linker remains with N terminal cytosol tail N terminal transmembrane domain removed, and an IgK signal sequence was used to place the remaining sequence in the correct orientation to be present in the lumen of the Golgi, and 5) G0069-dictyostelium peptide linker and C-terminal transmembrane domain removed.

Transfections were performed in Expi293 cells of the following sequences G0047, G0051, G0067, G0068, G0069. These constructs were transfected alone or with HPC4-tagged GBA construct (SEQ ID NO:9).

(01DO - IgK-HPC4-GBA) (K360N)
SEQ ID NO: 9
METDTLLLWVLLLWVPGSTGDEDQVDPRLIDGKGSRPCIPKSFGYS
SVVCVCNATYCDSFDPPTFPALGTFSRYESTRSGRRMELSMGPIQ
ANHTGTGLLLTLQPEQKFQKVKGFGGAMTDAAALNILALSPPAQN
LLLKSYFSEEGIGYNIIRVPMASCDFSIRTYTYADTPDDFQLHNF
SLPEEDTKLKIPLIHRALQLAQRPVSLLASPWTSPTWLKTNGAVN
GKGSLKGQPGDIYHQTWARYFVKFLDAYAEHKLQFWAVTAENEPS
AGLLSGYPFQCLGFTPEHQRDFIARDLGPTLANSTHHNVRLLMLD
DQRLLLPHWAKVVLTDPEAAKYVHGIAVHWYLDFLAPANATLGET
HRLFPNTMLFASEACVGSKFWEQSVRLGSWDRGMQYSHSIITNLL
YHVVGWTDWNLALNPEGGPNWVRNFVDSPIIVDITKDTFYKQPMF
YHLGHFSKFIPEGSQRVGLVASQKNDLDAVALMHPDGSAVVVVLN
RSSKDVPLTIKDPAVGFLETISPGYSIHTYLWRRQ

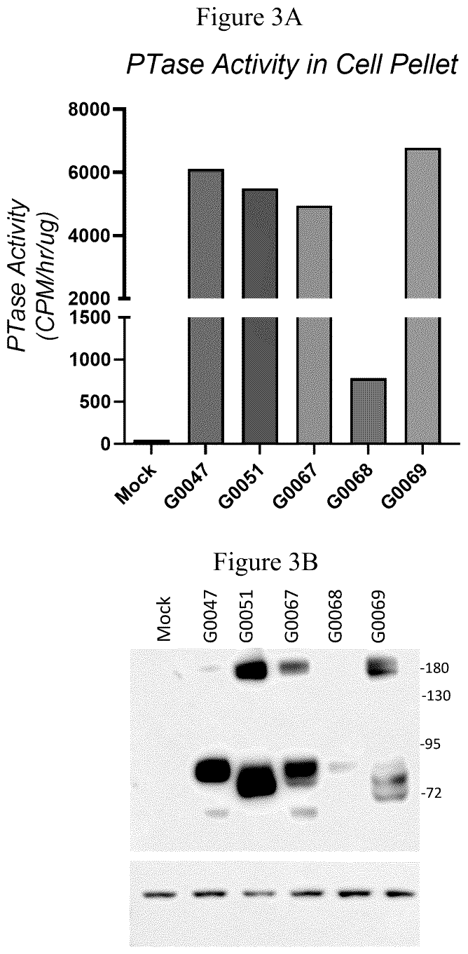

Cell lysates for the cells transfected with the S1S3 constructs alone were examined for PTase activity as described above. FIG. 3A shows that PTase activity was detected for all constructs. FIG. 3B is a Western blot from the transfected cells probed with an anti-V5 tag antibody to detect S1S3 enzyme expression. The data further confirmed that the removal of D-Dicty linker sequence does not alter S1S3 PTase activity. Also, the S1S3 variant without C-terminal transmembrane and cytosol domain maintains similar S1S3 activity as the original S1S3. The data also suggests the N-terminal transmembrane and cytosol domain is important for enzyme activity and localization.

### Example 5

### S1S3 PTase Variant Ability to Phosphorylate Lysosomal Enzyme GCase.

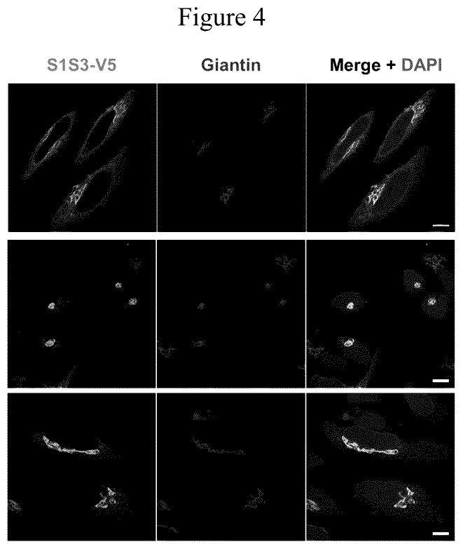

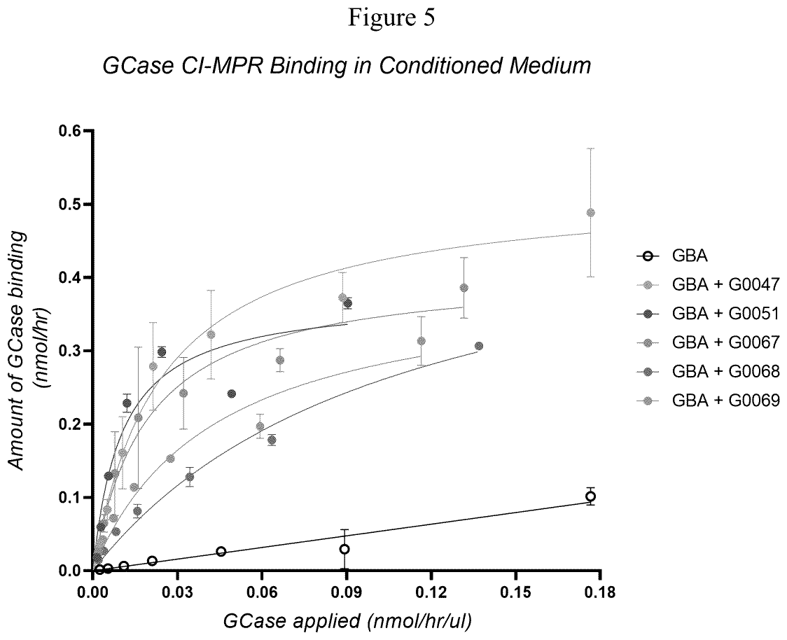

GBA1 gene, mutation of which will lead to Gaucher disease, was co-transfected with the S1S3 variants described in Example 4 (See FIG. 2). Transfections were performed in Expi293 cells of the same sequences G0047, G0051, G0067, G0068, G0069 that were co-transfected with an HPC4 tagged GBA1 construct. Conditioned media from transfected cells was used for a CI-MPR binding study. Binding of GBA protein was assessed using a GCase activity assay. As shown in FIG. 4, all constructs for S1S3 (G0047, G0051, G0067, G0068, G0069) enhanced the binding of the GCase enzyme expressed by the GBA1 gene to the CI-MPR.

### Example 6

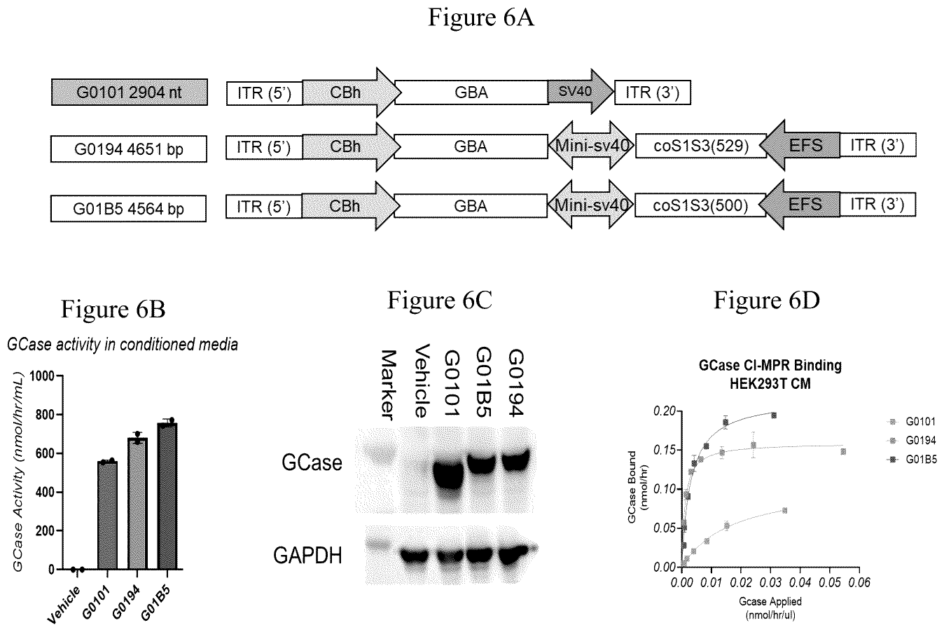

Plasmids for AAV packaging were transfected in HEK293T cells to assess GCase (GBA1 gene) and S1S3 expression and M6P phosphorylation. A schematic representation of the plasmids that were transfected are shown in FIG. 6A, designated as G0101, G0194 and G01B5. In the construct designated G0194, the coS1S3 (529) is G0067 (SEQ ID. NO. 6), and in the construct designated G01B5, the coS1S3 (500) is G0069 (SEQ ID. NO. 8). The GBA sequence used in constructs G0194 and G01B5 has the polynucleotide sequence shown as SEQ ID NO. 5 in WO2021/003442A. In all constructs, on the top strand, the Cbh promoter drives GBA expression. In G0194 and G01B5, the reverse complement strand also has an EFs promoter to drive codon-optimized S1S3 expression for modified S1S3 with 529 amino acids (G0194, coS1S3 (529)) or 500 amino acids (G01B5, coS1S3 (500)). coS1S3 (529) contains the N-transmembrane domain and 29 amino acid linkers, while coS1S3 (500) contains only the N-transmembrane domain with no linker.

48 h after transfection, conditioned media and cell lysates were collected. A GCase activity assay was performed on conditioned media as described in WO2021/003442A1. The results, shown graphically in FIG. 6B, demonstrate that G0101, G0194 and G01B5 all express comparable amounts of GCase activity, which is greatly enhanced compared to vehicle transfected cells with minimal detectable GCase activity.

GCase expression was also examined by Western blot for cell lysate from transfected cells (FIG. 6C). G0101, G01B5 and G0194 all show strong GCase expression compared to vehicle transfected cells.

Next, the conditioned media from transfected cells was analyzed for binding of GCase to CI-MPR on a plate binding assay as described in WO2021/003442A1. Binding was detected by a standard GCase activity assay. The results, shown in FIG. 6D, show that both G01B5 and G0194 show enhanced binding to CI-MPR compared to GCase expressed without S1S3 (G0101). These results demonstrate that both removal of the C-transmembrane or dictyostelium linker sequence retain S1S3 functionality.

### Example 7

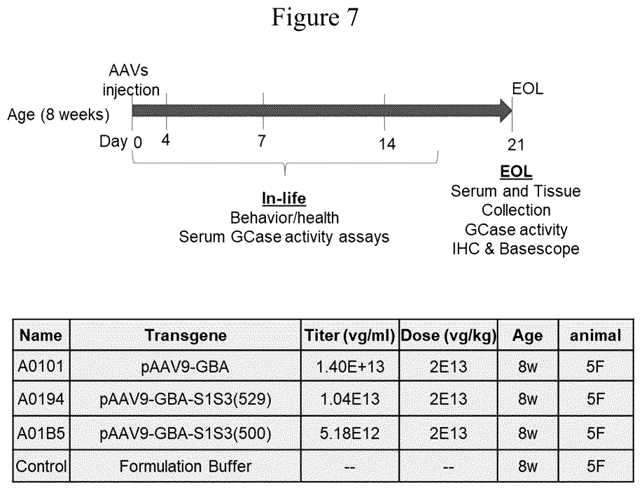

AAV9 were produced for in vivo study using the G0101, G0194 and G01B5 packaging plasmids described in Example 6 to make AAV constructs, designated A0101, A0194 and A01B5. (SAB Tech., Inc.). To access the tissue distribution in small animals, 8-week-old wild type mice received an intravenous injection of 2E13 VG/kg of A0101, A0194 or A01B5 AA9, or a formulation buffer control. Serum was collected for mice on 0, 4, 7, 14 or 21 days after injection. On day 21, tissues were also collected for analysis (brain, heart, liver, spleen, lung, bone marrow). Serum was analyzed for GCase activity, and tissues were analyzed for GCase activity, gene copy and transcription analysis according to the protocol shown in FIG. 7. At EOL, tissue from Brain, Heart, Liver, Spleen, Lung, Bone marrow (consistent regions) collected. In-life behavior/health was analyzed. GCase activity assay performed on all serum samples, tissue samples. Gene copy and transcription analysis in tissue samples.

For gene copy and transcription analysis, tissue sections (liver, heart, brain, bone marrow, spleen) were homogenized and DNA and mRNA extracted using a column purification kit (Zymo (Zymo DNA/RNA mini kit D7003). Gene copy and mRNA expression were examined by ddPCR. For gene copy, GBA and S1S3 were quantified using gene specific primers relative to mouse mTERT gene. For mRNA expression, GBA and S1S3 were quantified using gene specific primers relative to mouse ACTB gene.

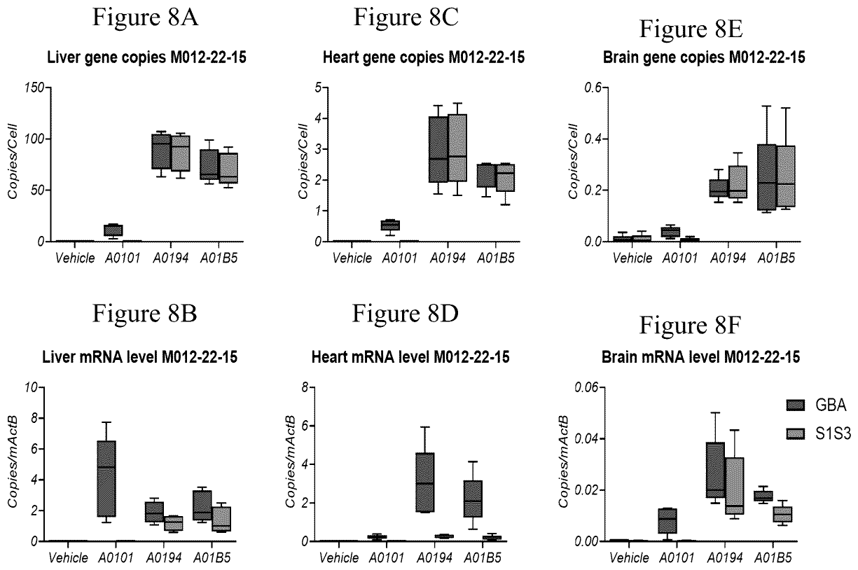

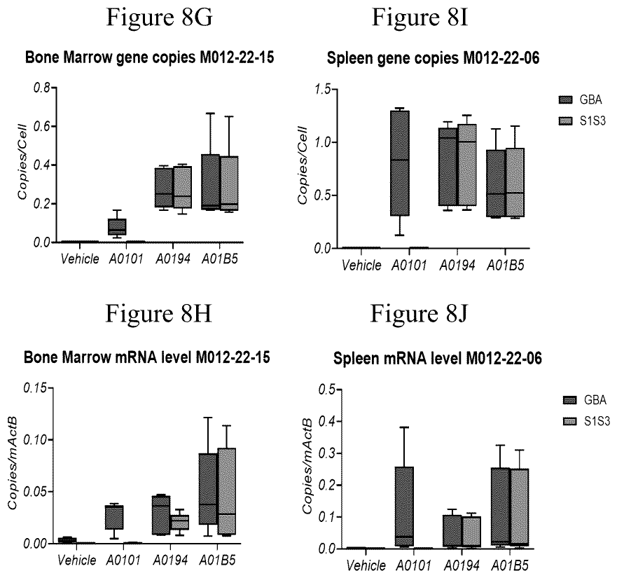

The results, shown graphically in FIGS. 8A-8J, demonstrate that GBA gene transduction and mRNA expression are present for all 3 virus constructs (A0101, A0194 and A01B5). S1S3 gene copy and mRNA expression are only detectable for A0194 and A01B5. Genes and mRNA were detectable in all tissues examined.

### Example 9

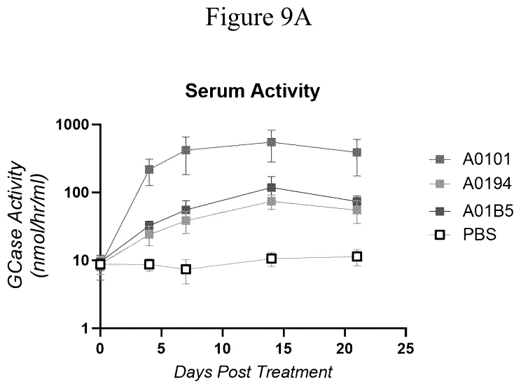

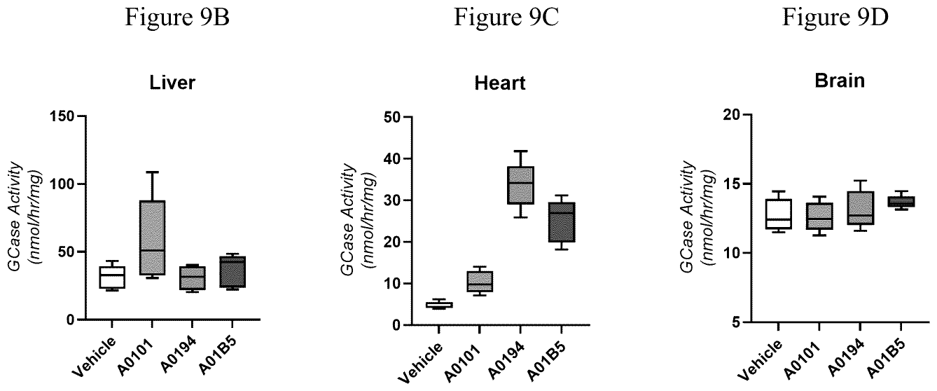

Total GCase produced in serum for wild type mice transduced with vehicle, A0101, A0194 and A01B5 was measured according to the following protocol. Serum was collected 0, 4, 7, 14 or 21 days after injections, and analyzed by a 4MU GCase activity assay. The results, shown graphically in FIG. 9A demonstrate that AAV transduced mice show enhanced GCase activity compared to vehicle treated mice.

Tissues from the liver, heart and brain from the AAV9 transduced mice were harvested 21 days post injection and homogenized. GCase activity was examined by 4MU activity assay. The results demonstrate that animals treated with A01B5 AAV9 show increased GCase level in liver (FIG. 9B), heart (FIG. 9C), and brain (FIG. 9D).

### Example 10

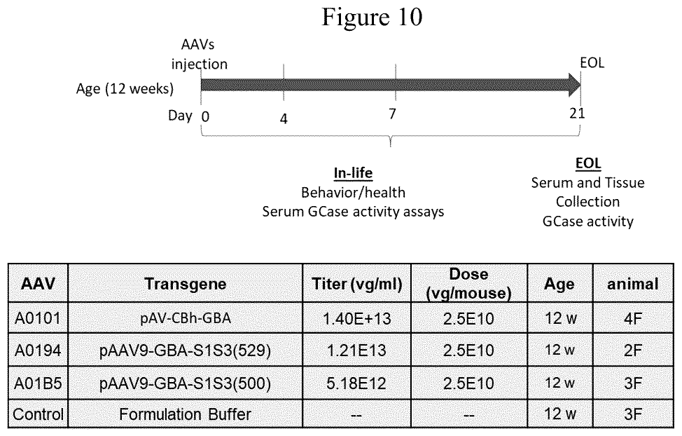

To explore the biodistribution of the AAV constructs in the brain, 12-week-old wild type mice received an intracisternal magna (ICM) injection of 2.5E10 vg/mouse of AAV9 constructs A0101, A0194 or A01B5, or a formulation buffer as control according to the protocol shown in FIG. 10. Brain tissues were collected 21 days after injection. Mice were examined by BaseScope to examine GBA gene mRNA level in brain and IHC staining for GCase protein distribution in brain. Tissue sections were taken from mouse brain sections. Fluorescence microscopy was performed to examine the distribution of human GCase protein and human GBA mRNA for animals treated with AAV9 by ICM injection. Mice in this study were C57B6/J. Route for treatment, ICM. Time points for collection was 3 weeks for the expression/biodistribution study. EOL collection of brain. In-life behavior/health was analyzed. Brain kept at Sanford for IHC/basescope staining. Different regions of brain were stained for GCase to look at distribution following ICM injection. RNAScope/BaseScope performed for hGBA in brain sections.

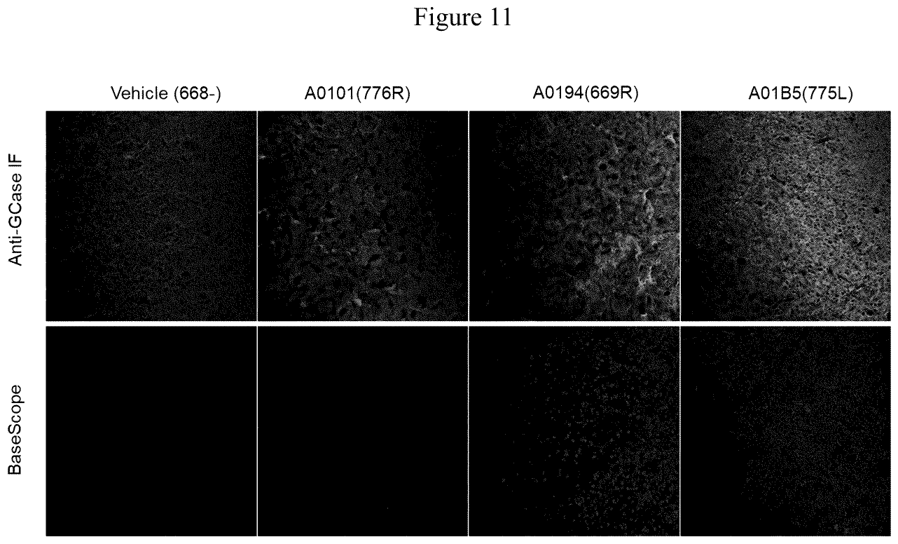

The results are shown in FIG. 11: the top panels show increased human GCase protein in mouse brain sections detected by anti-human GCase antibody. Stronger GCase signal was detected in the A0194 and A01B5 AAV9 treated animals. In the bottom panels, BaseScope was performed with human GBA mRNA specific probes to examine distribution of human GBA gene from transduced cells. The images show higher levels of GBA mRNA in A0194 and A01B5 treated animals.

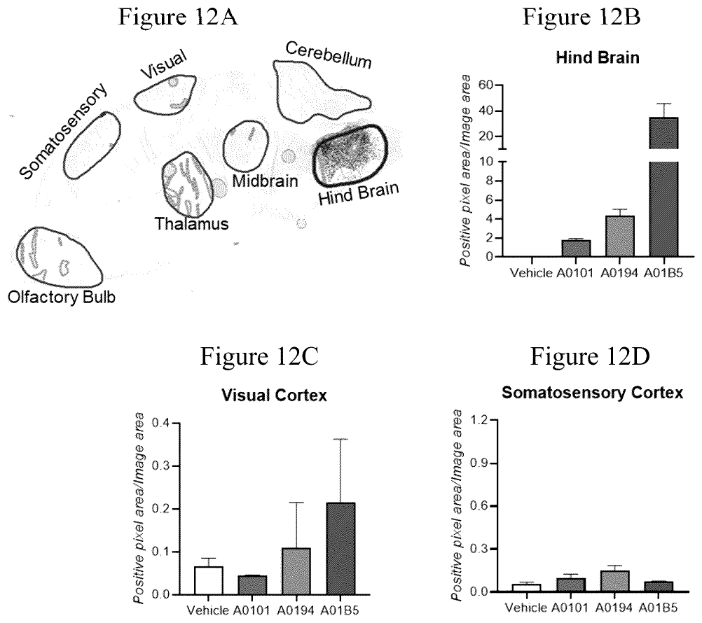

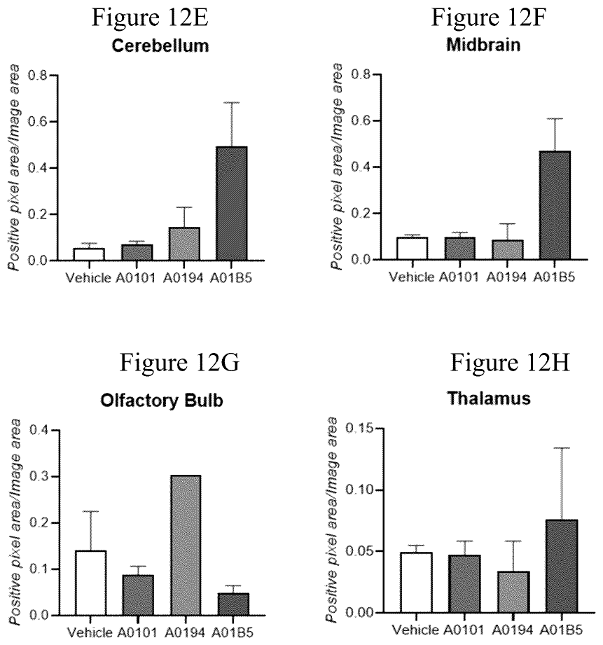

In addition, mouse brain sections were stained by immunohistochemistry for the presence of human GCase protein. Sections were quantified for GCase protein expression as shown in FIG. 12A: visual cortex, cerebellum, thalamus, midbrain or hind brain. Pixel intensity for human GCase positive staining was quantified (Image J) and plotted on the graphs shown in FIGS. 12B-12H for hind brain, visual cortex, somatosensory cortex, cerebellum, midbrain, olfactory bulb, and thalamus, respectively. The results show that enhanced staining of human GCase protein was detected in A0194 and A01B5 treated mouse brain regions.

1. A composition comprising a modified GlcNAc-1-phosphotransferase, wherein the modified GlcNAc-1-phosphotransferase comprises the amino acid sequence of SEQ ID NO: 1 with a deletion of amino acids 109-137.

2. The composition of claim 1, wherein the sequence of the modified GlcNAc-1-phosphotransferase comprises the amino acid sequence of SEQ ID NO: 2.

3. The composition of claim 1, wherein the deletion of amino acids 109-137 is replaced with an amino acid sequence of 5-6 amino acids.

4. The composition of claim 3, wherein the 5-6 amino acids comprises at least three glycines.

5. The composition of claim 3, wherein the amino acid sequence of 5-6 amino acids comprises glycine-glycine-glycine-glycine-serine, or glycine-serine-glycine-serine-glycine-serine.

6. (canceled)

7. The composition of claim 3, wherein the amino acid sequence of the modified GlcNAc-1-phosphotransferase comprises the amino acid sequence of SEQ ID NO: 3 or SEQ ID NO: 4.

8. The composition of claim 1, wherein the deletion of amino acids 109-137 is replaced with an amino acid sequence of 26 amino acids.

9. The composition of claim 8, wherein the amino acid sequence comprises at least ten glycines.

10. The composition of claim 8, wherein the amino acid sequence of the modified GlcNAc-1-phosphotransferase comprises the amino acid sequence of SEQ ID NO: 5.

11. The composition of claim 1, wherein the modified GlcNAc-1-phosphotransferase further comprises a deletion of the C-terminal transmembrane and cytosolic domain.

12. The composition of claim 11, wherein the deletion of the C-terminal transmembrane and cytosolic domain comprises a deletion of amino acids 530-576 of SEQ ID NO: 1.

13. The composition of claim 11, wherein the amino acid sequence of the modified GlcNAc-1-phosphotransferase comprises the amino acid sequence of SEQ ID NO: 8.

14. A composition comprising a modified GlcNAc-1-phosphotransferase, wherein the modified GlcNAc-1-phosphotransferase comprises the amino acid sequence of SEQ ID NO: 1 with a deletion of the C-terminal transmembrane and cytosolic domain.

15. The composition of claim 14, wherein the deletion of the C-terminal transmembrane and cytosolic domain comprises a deletion of amino acids 530-576 of SEQ ID NO: 1.

16. The composition of claim 14, wherein the amino acid sequence of the modified GlcNAc-1-phosphotransferase comprises the amino acid sequence of SEQ ID NO: 6 or SEQ ID NO: 8.

17. The composition of claim 14, wherein the modified GlcNAc-1-phosphotransferase further comprises a deletion of amino acids 109-137 of SEQ ID NO: 1.

18. The composition of claim 17, wherein the sequence of the modified GlcNAc-1-phosphotransferase comprises the amino acid sequence of SEQ ID NO: 8.

19. A composition comprising a vector comprising a sequence encoding a first polynucleotide and a second polynucleotide encoding a modified GlcNAc-1 phosphotransferase (GlcNAc-1 PTase), wherein the modified GlcNAc-1 PTase comprises the composition of claim 1.

20. The composition of claim 19, wherein the vector is an adeno-associated viral (AAV) vector.

21. (canceled)

22. The composition of claim 19, wherein the modified GlcNAc-1-phosphotransferase further comprises a deletion of the C-terminal transmembrane and cytosolic domain; wherein the deletion of the C-terminal transmembrane and cystosolic domain comprises a deletion of amino acids 530-576 of SEQ ID NO: 1.

23. (canceled)

24. The composition of claim 19, wherein the first polynucleotide encodes a lysosomal protein.

25. A composition comprising a vector comprising a sequence encoding a first polynucleotide encoding a lysosomal enzyme and a second polynucleotide encoding a modified GlcNAc-1 phosphotransferase (GlcNAc-1 PTase), wherein the modified GlcNAc-1 PTase comprises the composition of claim 14.

26. The composition of claim 25, wherein the vector is an adeno-associated viral (AAV) vector.

27. (canceled)

28. The composition of claim 25, wherein the deletion of the C-terminal transmembrane and cytosolic domain comprises a deletion of amino acids 530-576 of SEQ ID NO: 1.

29. The composition of claim 25, wherein the modified GlcNAc-1-phosphotransferase further comprises a deletion of amino acids 109-137 of SEQ ID NO: 1.

30. The composition of claim 25, wherein the first polynucleotide encodes a lysosomal protein.
